(19) United States
(12) Patent Application Publication
(10) Pub. No.: US 2004/0235171 A1
(43) Pub. Date: Nov. 25, 2004

(54) Silencing of gene expression by sirna

(76) Inventor: Ann Josephine Milner, Park Road, Wilberfoss (GB)

Correspondence Address: GRAY CARY WARE FREDENRICH, 1625 MASSACHUSETTS AVENUE, NW, SUITE 300, WASHINGTON, DC, 20036-2247, US

(21) Appl. No.: 10/484,101

(22) PCT Filed: Jul. 17, 2002
(86) PCT No.: PCT/GB02/03300

(30) Foreign Application Priority Data
Jul. 17, 2001 (GB) 0117358.2
Jan. 14, 2002 (GB) 0200688.0
Jun. 17, 2002 (GB) 0213855.0

(51) Int. Cl. C07H 21/02; C12N 15/86

(52) U.S. Cl. 435/456; 536/23.1

(57) ABSTRACT

The present invention relates to a A method of selective post-transcriptional silencing in a mammalian cell of the expression of an exogenous gene of viral origin is provided. The method involves comprises introducing an siRNA construct into a mammalian cell where the siRNA construct is homologous to a part of the mRNA sequence of the exogenous gene. The invention also comprises an An siRNA construct with a nucleotide sequence which is homologous to a part of the mRNA sequence of an exogenous gene of viral origin and to the use of such a construct as a medicament also are provided.

### FIELD OF THE INVENTION

This invention relates to the application of siRNAs to silence gene expression.

### BACKGROUND TO THE INVENTION

Post-transcriptional silencing of eukaryotic genes can be achieved by the introduction into cells of dsRNA homologous for the gene to be silenced (reviewed in Plasterk & Fenning, 2000; Sharp, 2001; Carthew, 2001; Bass, 2001). Silencing is effected at several levels, including the selective targeting and degradation of the homologous mRNA. The RNA interference (RNAi) is sub-stoichiometric such that a vast excess of cellular mRNA is completely and selectively destroyed. Moreover, in some systems RNAi can maintain selective gene silencing throughout a 50- to 100-fold increase in cell mass (see Carthew, 2001).

The current model for the mechanism of RNAi is based upon the observation that the introduced dsRNA is bound and cleaved by endonuclease RNase III to generate 21- and 22-nucleotide products. These small interfering RNAs (siRNAs) remain stably complexed with the endonuclease. The resulting dsRNA-protein complexes appear to represent the active effectors of selective degradation of homologous mRNA (Hamilton & Baulcombe, 1999; Zamore et al., 2000; Elbashir et al., 2001a). Indeed, it has been established that duplexes of 21-nucleotide RNAs are sufficient to suppress expression of endogenous genes in mammalian cells (Elbashir et al., 2001b). This was demonstrated by selective silencing of endogenous lamin A/C expression in human epithelial cells following introduction of the cognate siRNA duplex. Thus, introduction of siRNA into mammalian cells is sufficient to selectively target homologous mRNA and silence gene expression. Importantly, siRNAs do not induce the non-specific interferon response, observed with dsRNAs >30 nucleotides long (Minks et al., 1979).

### STATEMENT OF THE INVENTION

According to the present invention there is provided a method of selective post-transcriptional silencing in a mammalian cell of the expression of an exogenous gene of viral origin comprising introducing into said mammalian cell an siRNA construct which is homologous to a part of the mRNA sequence of said gene.

In a preferred method of the invention the gene is present in the mammalian cell prior to the introduction of said siRNA.

In a further preferred method of the invention said nucleotide sequence is homologous to an unbroken or contiguous mRNA sequence of said gene.

In a yet further preferred method of the invention the exogenous gene of viral origin is any gene which causes disease in the mammalian cell.

As used herein the term ‘disease’ is used to refer to any abnormal or unhealthy condition of the body (or part of it) or of the mind.

In a further preferred method of the invention the exogenous gene is any oncogene of viral origin.

Preferably said oncogene is encoded by a papilloma virus, preferaby a human papilloma virus (HPV).

Human papillomaviruses vary in their pathological effects. For example, in humans so called low risk HPVs such as HPV-6 and HPV-11 cause benign hyperplasias such as genital warts, (also referred to as condyloma acuminata) while high risk HPVs, for example, HPV-16, HPV-18, HPV-31, HPV-33, HPV-52, HPV-54 and HPV-56, can cause cancers such as cervical or penile carcinoma HPV-16 and HPV-18 are causually linked to cervical cancer. HPV-1 causes verruca vulgaris. HPV-5 and HPV-8 cause malignant squamous cell carcinomas of the skin. HPV-2 is found in malignant and non malignant lesions in cutaneous (skin) and squamous (oral) epithelium.

Preferably the oncogene is the HPV E7 gene.

In an alternative embodiment of the invention, the oncogene is the HPV E6 gene.

In a preferred embodiment of the invention said siRNA is derived from a nucleic acid molecule selected from the group consisting of:

i) a nucleic acid molecule as represented by any nucleic acid sequence in FIG. 11;

ii) a nucleic acid molecule which hybridizes to any of the nucleic acid sequences in (i) and which has siRNA activity, and

iii) a nucleic acid molecule which is degenerate as a result of the genetic code to any of the nucleic acid sequences of (i) and/or (ii) above.

The present invention also provides an siRNA construct having a nucleotide sequence which is homologous to a part of the mRNA sequence of an exogenous gene of viral origin.

In a preferred embodiment of the invention said siRNA construct comprises a nucleic acid molecule, or part thereof, which encodes at least part of an oncogene wherein said nucleic acid molecule is selected from the group consisting of:

i) a nucleic acid molecule as represented by any nucleic acid sequence in FIG. 11;

ii) a nucleic acid molecule which hybridizes to any of the nucleic acid sequences in (i) and which has siRNA activity;

iii) a nucleic acid molecule which is degenerate as a result of the genetic code to any of the nucleic acid sequences of (i) and/or (ii) above.

In a preferred embodiment of the invention said nucleic acid molecule hybridizes under stringent hybridization conditions.

Typically, hybridization conditions uses 4-6×SSPE (20×SSPE contains 175.3 g NaCl, 88.2 g NaH₂PO₄H₂O and 7.4 g EDTA dissolved to 1 litre and the pH adjusted to 7.4); 5-10× Denhardts solution (50× Denhardts solution contains 5 g Ficoll (type 400, Pharmacia), 5 g polyvinylpyrrolidone and 5 g bovine serum albumen; 100 μg-11.0 mg/ml sonicated salmon/herring DNA; 0.1-1.0% sodium dodecyl sulphate; optionally 40-60% deionised formamide. Hybridization temperature will vary depending on the GC content of the nucleic acid target sequence but will typically be between 42°-65°. It is well known in the art that optimal hybridization conditions can be calculated if the sequences of the nucleic acid is known. For example, hybridisation conditions can be determined by the GC content of the nucleic acid subject to hybridization. Please see Sambrook et al (1989) Molecular Cloning; A Laboratory Approach. A common formula for calculating the stringency conditions required to achieve hybridization between nucleic acid molecules of a specified homology is:

Tₘ=81.5° C.+16.6 Log[Na⁺]+0.41[% G+C]−0.63 (% formamide).

Preferably the degree of homology is at least 75% sequence identity, preferably at least 85% identity; at least 90% identity, at least 95% identity; at least 97% identity, or at least 99% identity.

In an alternative preferred embodiment of the invention the RNAi molecule is between 15 bp and 25 bp, more preferably said molecule is 21 or 22 bp. Most preferably said molecule is less than 22 bp.

In a preferred embodiment of the invention said construct is part of a vector.

Preferably said vector is an expression vector adapted for expression of said siRNA. siRNA's may be manufactured recombinantly or by oligonucleotide synthesis. In the former vectors are adapted by the provision of promoters which synthesize sense and antisense molecules followed by annealing of molecules to form the siRNA molecule.

In yet a further preferred embodiment of the invention said siRNA molecules comprise modified nucleotide bases.

It will be apparent to one skilled in the art that the inclusion of modified bases, as well as the naturally occuring bases cytosine, uracil, adenosine and guanosine, may confer advantageous properties on siRNA molecules containing said modified bases. For example, modified bases may increase the stability of the siRNA molecule thereby reducing the amount required to produce a desired effect. The provision of modified bases may also provide siRNA molecules which are more or less stable.

The term “modified nucleotide base” encompasses nucleotides with a covalently modified base and/or sugar. For example, modified nucleotides include nucleotides having sugars which are covalently attached to low molecular weight organic groups other than a hydroxyl group at the 3′ position and other than a phosphate group at the 5′ position. Thus modified nucleotides may also include 2′ substituted sugars such as 2′-O-methyl-; 2-O-alkyl; 2-O-allyl; 2′-S-alkyl; 2′-S-allyl; 2′-fluoro-; 2′-halo or 2;azido-ribose, carbocyclic sugar analogues a-anomeric sugars; epimeric sugars such as arabinose, xyloses or lyxoses, pyranose sugars, furanose sugars, and sedoheptulose.

Modified nucleotides are known in the art and include by example and not by way of limitation; alkylated purines and/or pyrimidines; acylated purines and/or pyrimidines; or other heterocycles. These classes of pyrimidines and purines are known in the art and include, pseudoisocytosine; N4, N4-ethanocytosine; 8-hydroxy-N6-methyladenine; 4-acetylcytosine, 5-(carboxyhydroxylmethyl) uracil; 5-fluorouracil; 5-bromouracil; 5-carboxymethylaminomethyl-2-thiouracil; 5-carboxymethylaminomethyl uracil; dihydrouracil; inosine; N6-isopentyl-adenine; 1-methyladenine; 1-methylpseudouracil; 1-methylguanine; 2,2-dimethylguanine; 2-methyladenine; 2-methylguanine; 3-methylcytosine; 5-methylcytosine; N6-methyladenine; 7-methylguanine; 5-methylaminomethyl uracil; 5-methoxy amino methyl-2-thiouracil; β-D-mannosylqueosine; 5-methoxycarbonylmethyluracil; 5-methoxyuracil; 2 methylthio-N-6-isopentenyladenine; uracil-5-oxyacetic acid methyl ester; psueouracil; 2-thiocytosine; 5-methyl-2 thiouracil, 2-thiouracil; 4-thiouracil; 5-methyluracil; N-uracil-5-oxyacetic acid methylester; uracil 5-oxyacetic acid; queosine; 2-thiocytosine; 5-propyluracil; 5-propylcytosine; 5-ethyluracil; 5-ethylcytosine; 5-butyluracil; 5-pentyluracil; 5-pentylcytosine; and 2,6,-diaminopurine; methylpsuedouracil; 1-methylguanine; 1-methylcytosine.

The siRNAi molecules of the invention can be synthesized using conventional phosphodiester linked nucleotides and synthesized using standard solid or solution phase synthesis techniques which are known in the art. Linkages between nucleotides may use alternative linking molecules. For example, linking groups of the formula P(O)S, (thioate); P(S)S, (dithioate); P(O)NR12; P(O)R′; P(O)OR6; CO; or CONR12 wherein R is H (or a salt) or alkyl (1-12C) and R6 is alkyl (1-9C) is joined to adjacent nucleotides through —O— or —S—.

The present invention also provides an siRNA construct or vector for use as a medicament.

The present invention also provides for the use of an siRNA for the manufacture of a medicament for the treatment of cancer, particularly human cervical cancer, HIV, smallpox, flu and the common cold.

In a preferred embodiment of the invention there is provided the use of an siRNA for the manufacture of a medicament for the treatment of a disease caused by a human papilloma virus.

In a preferred embodiment of the invention said disease is selected from the group consisting of: genital warts; cervical cancer; penile cancer; malignant squamous cell carcinomas; verruca vulgaris.

The present invention also provides a method of treatment comprising administering to a patient in need of such treatment an effective dose of siRNA.

The present invention also provides a pharmaceutical composition comprising an siRNA construct of the invention in combination with a pharmaceutically acceptable excipient.

Reference will be made hereinbelow to the selective silencing of the gene responsible for the production of the E6 protein of the human papilloma virus (HPV), thereby leading to p53 accumulation resulting in apoptosis of HPV-positive cervical carcinoma cells. Reference will also be made to the selective silencing of the gene responsible for the production of the E7 protein of HPV thereby leading to induced apoptotic cell death. However, the present invention may have application to many other diseases resulting from the introduction into mammalian cells of viral exogenous genes. Other examples include HV, CMV, flu, the common cold, smallpox and genes introduced during germ warfare.

### BRIEF DESCRIPTION OF THE DRAWINGS

An example to illustrate the present invention will be described below with reference to the accompanying drawings, in which:

FIG. 1 shows selected E6 siRNA based upon the position of its homologous sequence in the HPV16 E6 gene and its predicted RNA secondary structures. a, HPV16 E6 sequence (GenBank NC-001526) showing the positions of the E6 siRNA sequence (bold, underlined). b, five candidate HPV16 E6 siRNA sequences and their predicted potential for secondary structure formation. Sequence diversion from HPV18 E6 is indicated by bold, underlined nucleotides. The sequence chosen is indicated by an asterisk. c, Sequence of the control siRNA, non-homologous overall to HPV16 E6, although it contains a short sequence homologous with hpv16 e6 NTS 339 to 347. Such short homologies are known to be insufficient for dsRNA silencing (Elbashir et al, 2001b).

FIG. 2 illustrates the reduction caused by siRNA in HPV16 E6 mRNA levels in CaSKi cells. a, E6 mRNA levels revealed by Northern blotting of total mRNA purified from CaSKi cells at 24 hr post transfection with E6 siRNA, or 24 h after mock transfection. Results obtained with control siRNA were the same as those for mock-transfected cells. b, E6 and mRNA and p53 mRNA levels determined by RT-PCR at 15 hr and 24 hr post transfection with E6 siRNA and control siRNA, as indicated. Time 0 hr=non-transfected control cells at the start of the experiment. N/C=negative RT-PCR control without added total cellular RNA. Histograms in a and b show the relative amounts of HPV16 E6 mRNA (solid bars) and p53 mRNA (open bars) in each experiment as determined by gel scanning. c, E6 mRNA levels determined by semi-quantitative RT-PCR following serial dilutions of total cellular RNA samples as indicated. Samples were prepared at 0 hr, 15 hr and 24 hr post-transfection with either E6 siRNA or control siRNA as indicated. pSP6E6=HPV16 E6 cDNA plasmid, 1 pg starting concentration.

FIG. 3 E6 siRNA causes stabilisation of p53 protein in CaSKi cells. a, p53 protein immunoblot of lysate samples of cells transfected with E6 siRNA and harvested at 15 hr, 24 hr, 39 hr and 48 hr post-transfection as indicated. Time 0 hr=non-transfected cells at the start of the experiment. b, p53 mRNA levels as determined by RT-PCR. c, Separate experiment showing the level of p53 protein (i) in cells transfected with E6 siRNA relative to mock-transfected cells (solid line) and (ii) in cells transfected with control siRNA relative to mock transfected cells (dashed line). Protein gel loading was normalised to cell numbers and confirmed by Ponceau staining.

FIG. 4 Stabilisation of p53 by E6 siRNA correlates with up-regulation of p21, a p53 target gene. Samples from CaSKi cell lysates used for FIG. 3c were probed for p21. Immunoblots show p21 protein levels at various times post-transfection with a, E6 siRNA, b, control siRNA, and c, mock transfection without siRNA. Protein equivalence between samples was confirmed by actin levels.

FIG. 5 siRNA sequences and transfections efficiencies. a, siRNA sequences used in this study and their relative positions within HPV16 E6 and E7 mRNAs. Predicted secondary structures (dimers and loops) were derived using Vector NTI. b, Transfection efficiencies (means of triplicates) obtained for each cell line used in this study.

FIG. 6 E6 siRNA and E7 siRNA induce selective loss of E6 and E7 mRNAs respectively. a, quantitiation of mRNA by Northern blotting and b, by semi-quantitative RT-PCR gave similar results. Results shown are for control siRNA and E6 siRNA at 48 hr. c-e, Cells analysed at 0, 24 and 48 hours after treatment with different siRNA as indicated. Results obtained for CaSki and SiHa were essentially identical; a and b, are examples of CaSki and c-e, are examples of SiHa cells. Viral E6 and E7 mRNAs, and cellular p53 mRNA are identified above the historgrams (c-e).

FIG. 7 Treatment with E6 siRNA induces activation of cellular p53 protein. a, SiHa cells treated with E6 siRNA show marked increase in p53 protein accompanied by p21 expression, as determined by immunoblotting. Parallel transfections with b, E7 siRNA or c, control siRNA fail to induce similar effects on p53 and p21 proteins. Similar results were obtained for CaSki cells. Equivalent sample loading for immunoblots was confirmed in every case by actin levels, as shown in d, for E6 siRNA-treated samples.

FIG. 8 E6 siRNA induces nuclear accumulation of p53 protein. Cells treated with control siRNA and E6 siRNA stained with Heochst for nuclei and with DO-1 antibody for p53 as indicated. CaSki cells 48 hour after transfection are shown, similar results were obtained for SiHa cells.

FIG. 9 E7 siRNA induces selective loss of hyper-phosphorylated cellular pRb. Lysates from SiHa cells treated with control siRNA, E6 siRNA or E7 siRNA were probed for pRb by immunoblotting. Rb*=hyper-phosphorylated pRb; Rb=hypo-phosphorylated pRb.

FIG. 10 Single dose E7 siRNA induces apoptosis in human cervical carcinoma cells. a-c, Phase contrast images of SiHa cells treated with control siRNA, E6 siRNA and E7 siRNA, as indicated. a, control siRNA has no effect on SiHa cell growth. b, E6 siRNA slows cell proliferation and at 96 hours islands of cells probably derived from non-transfected cells are visible. c, E7 siRNA induces apoptosis confirmed by f, FACS analysis of cells stained with annexin V. d, E7 siRNA does not affect growth of primary human normal diploid fibroblasts (NDF) nor of e, HCT116 colon carcinoma cells. Growth of NDF and HCT116 are also unaffected by control siRNA and E6 siRNA (not shown). f, control siRNA ( [symbol] ), E6 siRNA ( [symbol] ) and E7 siRNA (▪).

FIG. 11a, HPV 18 E6 b, HPV 18 E7 c, HPV 16 E6 d, HPV 16 E7 sequences.

### DETAILED DESCRIPTION OF THE INVENTION

Human carcinoma of the cervix is the second most common form of cancer in women worldwide. Over 90% of human cervical carcinomas are positive for the HPV which is a major risk factor for this disease. The cellular p53 tumour suppressor pathway is disrupted by HPV E6 which promotes uncontrolled degradation of p53. Selective inhibition of HPV E6 expression leads to p53 accumulation resulting in apoptosis of HPV-positive cervical carcinoma cells. Moreover, any agent which selectively targets intracellular HPV E6 is also selective at the cellular level, and only activates p53 in HPV-positive cells: normal cells and tissues would be unaffected.

Elevated levels of p53 are lethal and induce apoptosis in mammalian cells. The p53 protein is continually synthesised and degraded at high rates, resulting in a low steady state level in normal cells. Escape from degradation leads to rapid accumulation of activated p53 and apoptosis.

A major goal in cancer research is to activate p53 in tumour cells and, by this means, induce apoptosis of the malignant cell. Indeed, it is already established that activation of p53 is sufficient to induce apoptotic cell death in many tumours. Since most malignancies shut down p53 in order to survive, it follows that activation of p53 presents one of the most rewarding goals for novel anti-cancer therapies. Several approaches to the problem are being developed by various laboratories (see Woods & Vousden, 2001; Hupp et al., 2000). These include (i) re-introduction of p53 by gene therapy, (ii) pharmacological restoration of wild type protein conformation to mutant p53 using small molecules (see, for example, Foster et al., 1999), and (iii) metabolic stabilisation of wild type p53 by disruption of p53-hdm2 interaction. These and other approaches are reviewed in Woods & Vousden (2001) and Hupp et al., 2000).

As always, a major problem concerns selective targeting of tumour cells without adverse effects on normal, non-tumour cells. In the case of human cervical carcinoma, however, the involvement of HPV offers the possibility of selective targeting of the tumour cells via the oncogenic viral genes responsible for deregulated cell proliferation. HPV E6, in particular, is an attractive target for therapeutic intervention since E6 disrupts p53 function and causes uncontrolled degradation of p53 protein. Since p53 is constitutively expressed with high rates of synthesis, removal of its degradation leads to rapid accumulation of cellular p53 protein.

High risk types of human papilloma virus, HPV-16 and HPV-18, are causally linked with the development of around 90% cases of human carcinoma of the cervix. The HPV E6 protein of these high risk viruses plays a key role in the disruption of normal growth control and tumour suppressor pathways. HPV E6 complexes with cellular proteins p53 and E6-AP (a ubiquitin ligase) and causes uncontrolled degradation of p53 by the ubiquitin-dependent proteolytic system (Scheffner et al., 1990; Scheffner, 1998). In normal cells the rapid turnover of p53 protein is regulated by cellular hdm2 protein, which also targets p53 for degradation by the ubiquitin system (Haupt et al., 1997; Kubbutat et al., 1997). However, the hdm2 pathway for p53 degradation is switched off in HPV-positive cervical carcinoma cells (Hietanen et al., 2000). Thus the HPV E6/E6-AP pathway appears to be solely responsible for p53 degradation in HPV-positive cervical cancer carcinoma cells. Most HPV-positive human cervical carcinomas retain endogenous wild type p53 (see Woods & Vousden, 2001). By silencing HPV E6 this project aims to activate endogenous wild type p53, thereby initiating apoptosis in human cervical carcinoma cells.

Previous attempts to activate p53 in HPV-positive human cervical cancer cells have included (i) antisense RNA strategies (Steel et al., 1993), (ii) use of HPV E2 to repress E6 (Dowhanick et al., 1995); and (iii) use of leptomycin B, an inhibitor of nuclear export containing nuclear export signals, to cause nuclear accumulation of p53 in cervical carcinoma cells (Freedman and Levine, 1998). Combined treatment of human cervical carcinoma cell lines with leptomycin B plus actinomycin D reduces viral mRNA and activates p53-dependent apoptosis (Hietanen et al., 2000). However, all these approaches have major limitations in terms of leads towards therapeutic reagents. For example: antisense RNA strategies can be problematic and, at best, inefficient; and both leptomycin B and actinomycin D are highly toxic reagents.

The present invention represents a completely novel approach to activate p53 in human cervical carcinoma cells. E6 expression is altered and endogenous p53 is thereby activated in human cervical carcinoma cells. Normal cells are unaffected. Silencing of HPV E6 is achieved by exploiting recent advances in post-transcriptional gene silencing, using the phenomenon of RNA interference (RNAi).

The siRNAs are designed to target HPV E6 mRNA in human cervical carcinoma cells, using established cell lines. These novel siRNA reagents are then employed to silence E6 expression in the cervical carcinoma cells. Effects of E6 silencing on the p53 protein and upon cell growth and viability are monitored. Toxicity and specificity are assessed using normal, HPV-negative cell lines.

Specific Examples (1)

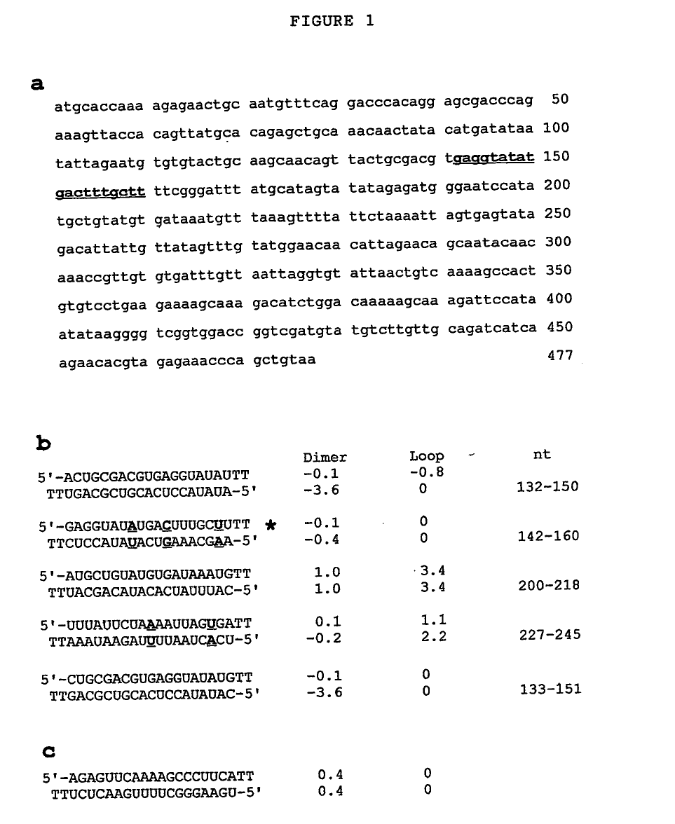

In order to demonstrate that siRNA can be employed to silence a viral oncogene of major importance in human cancer, namely the E6 gene of HPV16, CaSKi cells, a human cervical cancer cell line which contains approximately 600 tandem repeats of HPV16 integrated into the host cell genome were employed. The sequence of the HPV16 E6 gene is presented in FIG. 1a.

In choosing the RNA sequence with which to attempt E6 silencing, account was taken of (i) central positioning of the homologous sequence in the E6 mRNA, (ii) minimal potential for secondary RNA structure formation, and (iii) evolutionary conservation between the E6 genes of HPV16 and HPV18, both high risk types of human cervical cancer. Priority was given to central positioning combined with minimal theoretical secondary structure since both factors can affect the efficacy of RNA silencing (Elbashir et al, 2001a). The selected ds oligonucleotide is designated E6 siRNA (FIG. 1b, indicated by asterisk). As negative control (control siRNA; FIG. 1c) dsRNA of equivalent length and predicted secondary structure was employed. However, it lacked extensive homology to any part of the HPV E6 gene. Each base-paired 21-nucleotide (nt) RNA was synthesised with symmetric 2-nt 3′ overhangs composed of (2′-deoxy thymidine) since this may enhance nuclease resistance of siRNAs (Elbashir et all, 2001a and Elbashir et al, 2001b).

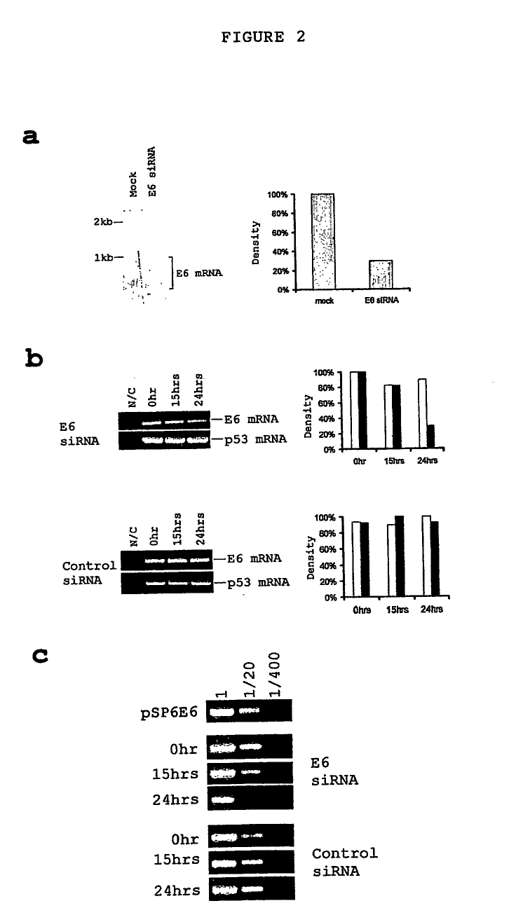

HPV16 viral gene expression is mediated by host cell transcription/translation machinery (zur Hausen, 2000). To test for silencing of viral E6 gene expression in CaSKi cells the levels of viral E6 mRNA were determined before and at various times after transfection with E6 siRNA. At 15 hr post transfection the level of E6 mRNA appeared to be unaffected by E6 siRNA, but by 24 hr there was a 70% reduction in the level of E6 mRNA as determined by Northern blotting (FIG. 2a). Similar results were observed using reverse transcription-polymerase chain reaction (RT-PCR, FIG. 2b upper panel) and semi-quantitative RT-PCR (FIG. 2c). The effect of E6 siRNA appeared to be specific since transfection with the 21-nt control siRNA had no effect on E6 mRNA levels (FIGS. 2b and 2c). Thus the loss of E6 mRNA is not due to a non-specific viral or cellular response to the introduction of short dsRNA molecules.

Further confirmation for the selectivity of E6 siRNA silencing was indicated by the levels of p53 mRNA which were unaffected following transfection with E6 siRNA (FIG. 2b). p53 mRNA levels were also unaffected by control siRNA (FIG. 2b). Moreover, cell growth and viability appeared to be unaffected up to 63 hr post-transfection with either E6 siRNA or the non-specific control siRNA (results not shown), indicating that the introduction of siRNA molecules into mammalian cells per se is non-toxic, and consistent with the observations of Elbashir et al. (2001a). Overall these results demonstrate that siRNA can selectively silence expression of a viral gene when it is stably integrated into the host mammalian cell genome.

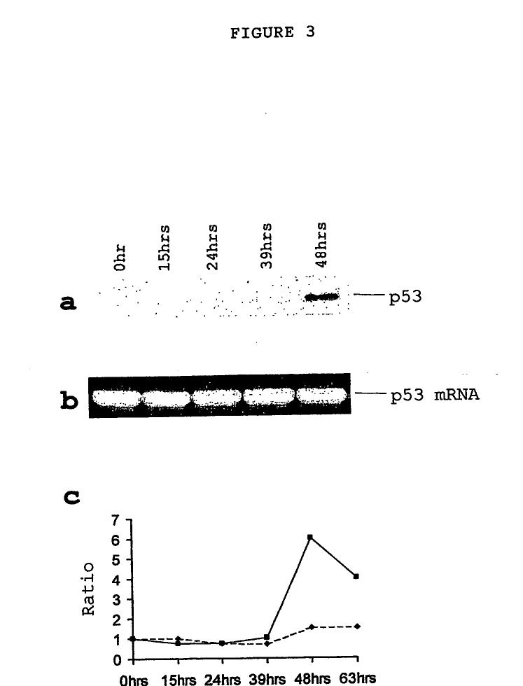

CaSKi cells express wild type p53 which, in normal cells, is subject to controlled degradation by Hdm2 (Levine, 1997). The levels of endogenous Hdm2 protein are very low in CaSKi and other HPV-positive human cervical cancer cell lines (Hietenan et al 2000) and the E6-mediated pathway appears to be solely responsible for p53 degradation in these cells (Hietenan et al, 2000 and Hengstermann 2001). Silencing of E6 expression should effectively abolish p53 degradation, resulting in increased levels of p53 protein in HPV-positive cells. To demonstrate this, prediction CaSKi cells were transfected with E6 siRNA and p53 protein levels were monitored over the subsequent 48 hr period, aiming to allow time for E6 mRNA degradation (see FIG. 2) plus turnover of pre-existing E6 protein. The levels of p53 were determined by immunoblotting. In non-transfected control cells p53 was barely detectable (FIG. 3a, time 0 hr). However, p53 protein levels began to increase 24 hour's post-transfection with E6 siRNA and continued to accumulate to 48 hours (FIG. 3a). It is to be noted that p53 mRNA levels remained constant over this 24 to 48 hr time period (FIG. 3b). The onset of p53 accumulation showed some variation and in two out of five experiments it occurred between 39 and 48 hrs post-transfection with E6 siRNA (see, for example, FIG. 3c, solid line). Transfection with non-specific control siRNA had no effect upon the level of p53 protein relative to mock-transfected cells (FIG. 3c, dashed line). This is an important control since p53 is a stress response protein and genotoxic stress can stabilise p53 in mammalian cells (Levine, 1997). Accordingly, it can be concluded (i) that siRNA alone is not sufficient to induce the stabilisation of p53 observed in CaSKi cells transfected with E6 siRNA (FIG. 3a), and (ii) that p53 stabilisation therefore reflects selective post-transcriptional silencing of the HPV E6 gene, with concomitant loss of E6-mediated targeting of p53 for uncontrolled degradation.

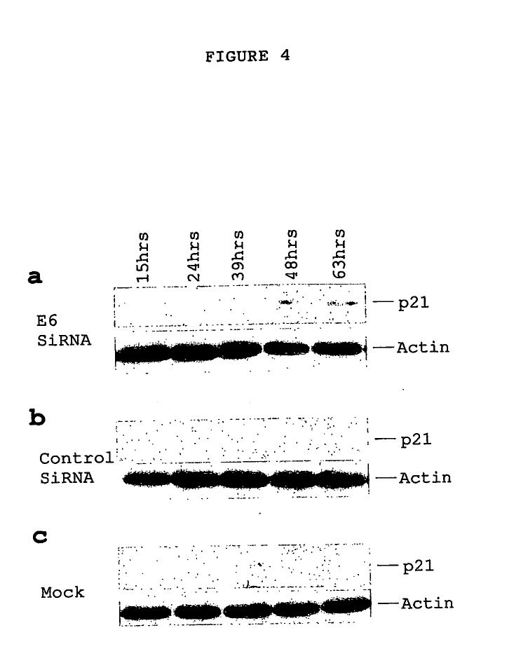

To determine whether the stabilised p53 protein is functionally competent following E6 silencing in HPV-positive cells. Its ability to up-regulate expression of the p21 protein was assessed. p21 is the product of a p53 target gene and is involved in p53-induced cell cycle arrest in normal cells (Levine,1997). Immunoblotting demonstrated that the p21 protein is very low or undetectable in CaSKi cells under normal conditions of growth, with levels equivalent to those observed 15 hrs post-transfection (see FIG. 4a). However, p21 became clearly detectable in cells transfected with E6 siRNA and a strong signal was first obtained 48 hr post-transfection (FIG. 4a), co-incident with p53 stabilisation in these cells (see FIG. 3c, solid line). In all experiments the induction of p21 correlated with stabilisation of p53 protein. In contrast, cells transfected with control siRNA (FIG. 4b), or mock transfected cells (FIG. 4c) showed no marked induction of p21 protein expression: this correlates with lack of p53 stabilisation in the cells. The most likely explanation for the observed up-regulation of p21 in the presence of E6 siRNA is that p53, protected from degradation by E6 silencing, retains wild type function and transactivates the p21 target gene. This is entirely consistent with earlier studies indicating that wild type p53 in HPV-positive cells retains its functional potential and that its residual activity is inversely proportional to the level of expressed HPV E6 (Butz, 1995, 1996 and 2000).

HPV E6 is a major player in the malignant transformation of human cervical carcinoma cells infected with high risk types of HPV (zur Hausen 2000). The oncogenic effects of HPV E6 have been shown to involve both p53-dependent and p53-independent pathways (zur Hausen 2000, Pim et al 1994, Pan et al 1994, Pan et al 1995, Liu et al 1999 and Thomas et al 1999). Continual expression of HPV E6, together with HPV E7, seems necessary for the maintenance of the malignant state in HPV-positive cells (von-Knebel-Doeberitz et al 1992). It follows that therapeutic intervention of HPV gene expression and/or viral protein function represents a prime objective in the development of novel strategies for the prevention and/or treatment of human cervical cancer (zur Hausen 2000, von Knebel-Doeberitz et al 1992, Hu et al 1995, Venturini et al 1999, Beer-Romero et al 1997 and Traidej et al 2000). Such a viral-targetted approach has the added bonus of tumour cell selectivity since only HPV-positive cells should be targeted with little, if any effect on normal cells and tissues. Thus the demonstration that siRNA has the ability to selectively silence HPV E6 expression identifies siRNA as a potent tool for the treatment of human cervical cancer.

The discovery that siRNA can be employed for selective silencing of viral gene expression within mammalian cells has far reaching implications. Application of siRNA should help elucidate key genes involved in viral pathogenesis. Moreover, both DNA and RNA viruses are likely to prove vulnerable to selective siRNA silencing, thus enabling the development of anti-viral therapies for diverse viral-induced diseases in humans and in other mammals.

Methods

RNA Preparation and mRNA Detection

21-nucleotide RNAs (FIG. 1) were synthesised and HPLC purified by GENSET SA (Paris, France). For annealing of the siRNAs, 20 μM single strands were incubated in annealing buffer (20 mM Tris-HCl pH7.5; 10 mM MgCl; and 50 mM NaCl) for 1 min at 90° C. followed by 1 hr at 37° C. For Northern blotting total mRNA was prepared using Oligotex (Qiagen) and run on a 1% agarose gel at room temperature under standard conditions. HPV16 E6 mRNA was detected using radiolabelled [³²P]-HPV E6 cDNA. All the RT-PCR reactions employed total RNA prepared using the RNeasy kit (Qiagen). For RT-PCR the Reverse-iT one-step kit (Advanced Biotechnologies) was employed. For E6 mRNA, the primers 5′cggaattcatgcaccaaaagagaactgca3′ and 5′cccaagcttacagctgggtttctctacg3′ were used in the thermal cycle: 47° C., 30 min; 94° C., 2 min; then 35 cycles of 94° C. 45 sec, 55° C. 45 sec and 72° C. 1 min; followed by 72° C. for 5 min. For p53 mRNA, the primers 5′atggaggagccgcagtcagat3′ and 5′tcagtctgagtcaggcccttc3′ were used, and the thermal cycle was as follows: 47° C., 30 min; 94° C., 2 min; then 35 cycles of 94° C. 45 sec, 58° C. 45 sec, 72° C. 2 min; and 72° C. 5 min. For semi-quantitative RT-PCR 100 ng total cellular RNA was diluted 1/20 and 1/400. Northern blots were repeated twice, and E6 and p53 RT-PCRs were repeated a minimum of four times with reproducible results.

Cell Culture and Transfection

CaSKi cells were maintained in RPMI plus 10% foetal calf serum (Life technologies), penicillin 100 units ml⁻¹ and streptomycin 100 μg ml⁻¹ at 37° C. in 5% CO₂ in air. Cell doubling time was approximately 24 h. For transfection cells were trypsinised and sub-culutred into 6 well plates (10 cm²) without antibiotics, 1.5×10⁵ cells per well. After 24 h the cells were transfected with siRNA formulated into liposomes (Oligofectamine, Life Technologies) according to the manufacturer's instructions. siRNA concentrations were 0.58 μg per well. The final volume of culture medium was 1.5 ml per well. Cells were harvested for analysis at various times thereafter as indicated in the results. Each experiment was carried out four or more times.

Immunoblotting

Transfected cells were trypsinised, washed in PBS and an aliquot removed for cell counting. The remaining cells were lysed in 50 μl lysis buffer (150 mM NaCl; 0.5% NP40; 50 mM Tris pH 8.0) on ice for 30 min. Samples were then diluted 1:1 in 4×strength Laemlli's sample buffer. (Residual insoluble proteins remaining in the cell pellets were taken up directly into Laemlli's buffer and also analysed but showed no significant differences between experimental and control cells; results not shown). Murine monoclonal antibody DO-1 was used to detect human p53 protein; and anti-p21 (SX118) (PharMingen) was used to detect p21 protein. Actin was detected using polyclonal antibody (Sigma). Note that it was not possible to monitor E6 protein levels in the transfected cells since there is no antibody available for its reliable quantitation. Equivalent amounts of total cellular protein were loaded, assessed either by Ponceau staining or by actin levels. Visualisation was carried out using BM enhanced chemiluminescence (Roche). Quantitation was by gel scanning of comparable, under-exposed signals.

Specific Examples (2)

Human papillomavirus (HPV) was selected as a clinically relevant viral target. High risk types of HPV are causally linked with initiation and malignant progression of human cervical carcinoma and encode at least three oncoproteins, namely E5, E6 and E7 (zur Hausen 2000, Thomas et al 1999, McMurray 2001). Of these E6 and E7 are best understood. For our studies we employed CaSKi and SiHa, two human cervical carcinoma cell lines positive for high risk type HPV16 and well characterised as models for the study of HPV-induced cell transformation (Hengstermann et al 2001, Butz et al 1995, Scheffner et al 1991, Butz et al 1996, Hietenan et al, 2000, Baker et al 1987). The E6 and E7 gene products of HPV are pleiotropic and appear to exert their transforming properties by binding, directly or indirectly, to cellular proteins linked with cell growth regulation (zur Hausen). Of particular importance are the interactions of E6 with p53, and E7 with the retinoblastoma protein (pRb). The p53 and pRb proteins are key tumour suppressors and cell cycle inhibitors in mammalian cells. Binding of E6 to p53 is mediated by E6-associated protein ligase (E6-AP) and targets p53 for ubiquitination and proteosomal degradation (Scheffner et al 1990, Scheffner et al 1993). E6 may decrease p53 capacity for growth inhibitory gene transactivation by suppressing the co-activators CBP and p300 (Patel et al 1999). In parallel, E7 binding to pRb results in hyper-phosphorylation of pRb and release of E2F transcription factors which activate genes for cell proliferation. Although HPV E6 and E7 can immortalise cells independently, their co-operative interactions substantially enhance immortalisation efficacy.

It was sought to silence HPV E6 and E7 gene expression and design siRNAs to target the respective viral mRNAs. The results indicate selective degradation of E6 and E7 mRNAs. Silencing was sustained for at least four days following a single dose of siRNA. E6 silencing induced accumulation of cellular p53 protein, transactivation of the cell cycle control p21 gene and reduced cell growth. In contrast, silencing of E7 induced apoptotic cell death. HPV-negative cells appeared unaffected by the anti-viral siRNAs. Thus we demonstrate for the first time (i) that siRNA can induce selective silencing of exogenous viral genes in mammalian cells, and (ii) that the process of siRNA interference does not interfere with the recovery of cellular regulatory systems previously inhibited by viral gene expression.

Choice of siRNA Sequences for Viral Gene Silencing

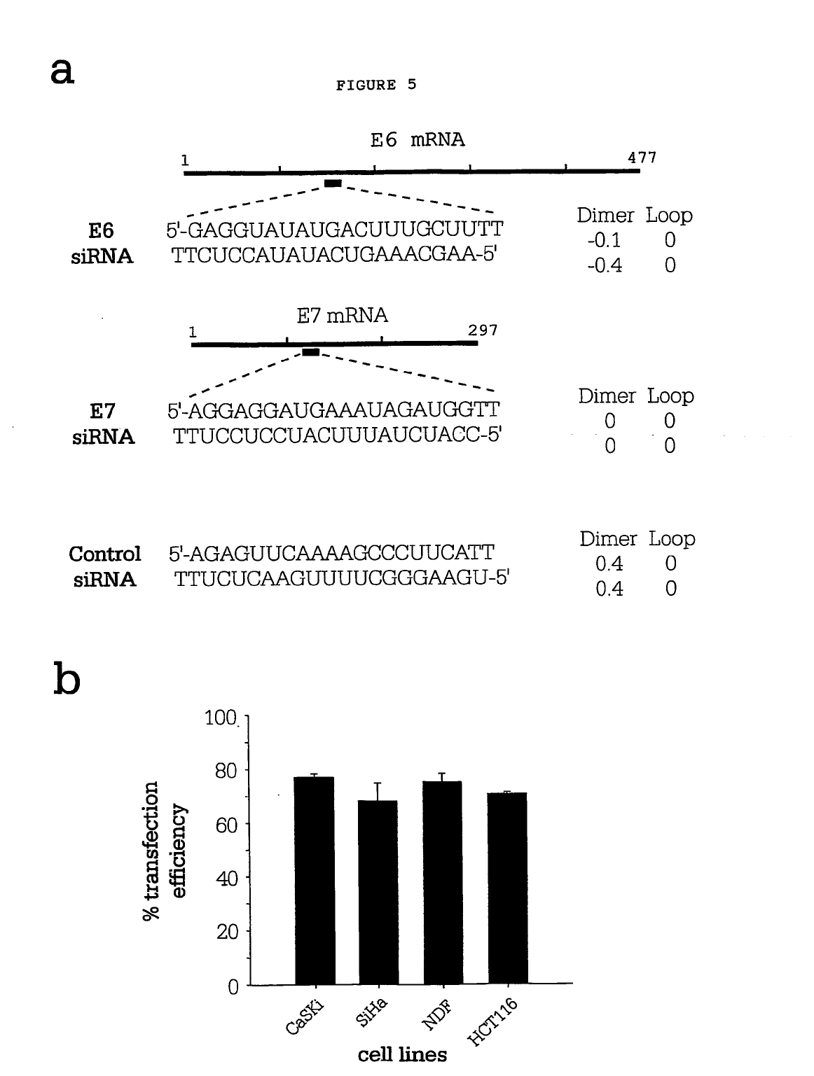

siRNA interference is influenced by secondary RNA structure and positioning of the cognate sequence within the intact mRNA molecule. The siRNAs chosen for this study are shown in FIG. 5a. Control siRNA was included in every experiment and lacks homology with HPV E6 and E7. None of the siRNAs share homology with exons of known human genes. Each 21-nucleotide (nt) RNA was synthesised with symmetric 2-nt overhang composed of (2′-deoxy thymidine) to enhance nuclease resistance. siRNA was introduced into cells by transfection (Materials and methods) and the transfection efficiency for each cell line is shown in FIG. 5b.

siRNA Causes Selective Loss of HPV E6 and E7 mRNAs

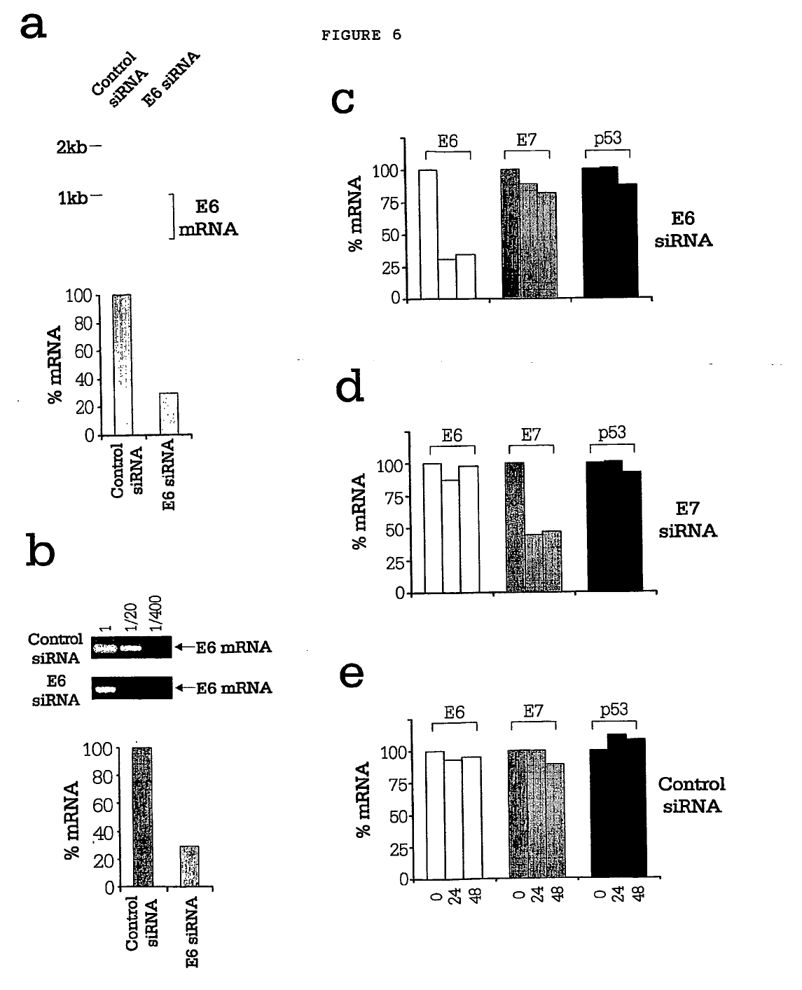

Little, if any change is viral mRNAs was observed in cells treated with control siRNA relative to non-treated controls. However, treatment with either E6 siRNA or with E7 siRNA induced a marked decrease in the respective E6 and E7 mRNA levels in both CaSki and SiHa cells (FIG. 6). Similar results were obtained by Northern blotting and by semi-quantitative RT-PCR, examples shown are for cells treated with control siRNA or E6 siRNA at 48 hr (FIGS. 6a and b). The decrease in E6 and E7 mRNA was maximal at 24 hours and was sustained for at least 4 days. Importantly, cellular p53 mRNA levels appeared unaffected under all conditions (FIG. 6c-e), indicating that anti-viral siRNAs do not activate generalised destruction of cellular mRNA. Approximately 70% reduction in E6 mRNA was observed following treatment with E6 siRNA (FIG. 6c). Since the transfection efficiencies were 70-80% (FIG. 5b) this represents close to complete loss of viral E6 mRNA in the transfected cells. In cells treated with E7 siRNA the reduction in E7 mRNA was approximately 50-60% (FIG. 6d). Selective targeting of the individual viral mRNAs were demonstrated by the following observations: (i) p53 mRNA levels were resistant to E6 and E7 siRNA treatment (FIGS. 6c and d); (ii) E6 mRNA levels were resistant to E7 siRNA and control siRNA (FIGS. 6d and e); and (iii) E7 mRNA levels were resistant to E6 siRNA and control siRNA (FIGS. 6c and e). Thus we conclude that treatment with E6 siRNA and E7 siRNA induces selective and differential degradation of the cognate viral E6 and E7 mRNAs in human cervical carcinoma cells.

Previous studies with mammalian cells have assessed siRNA silencing of endogenous genes at the level of the protein product (Elbashir et al 2001c, Caplen et al 2001, Harborth et al 2001, Kisielow et al 2002); the effects of siRNA on endogenous mRNA remain to be established. Our present results provide the first evidence that siRNA induces degradation of the target mRNA in mammalian cells (FIG. 6). Here the target was exogenous viral mRNA. A number of factors are likely to influence siRNA-induced degradation of mRNA (see Discussion) and it is interesting to note that, in both CaSKi and SiHa cells, the percentage reduction in E7 mRNA was consistently less than observed for E6 mRNA (approximately 40-50% versus 70%). Nonetheless, treatment of cells with either E6 siRNA or E7 siRNA induced the expected phenotypic responses (see below).

The Process of RNA Interference Does Not Adversely Affect Mammalian Cell Growth Regulatory Mechanisms: Activation of p53

If siRNA is to be developed as an experimental tool and/or for therapeutic applications it is important to establish that the process of RNA interference does not adversely affect cell control mechanisms. With this in mind we monitored cellular p53 protein in cells treated with siRNA. Both CaSKi and SiHa cells express wild type p53. In normal cells p53 levels are regulated by Hdm2-mediated degradation. However, Hdm2 is deficient in CaSKi and SiHa and the E6-mediated pathway is solely responsible for p53 degradation in these cells (Hengstermann et al 2001). Loss of E6 should therefore stabilise p53 protein in cells treated with E6 siRNA.

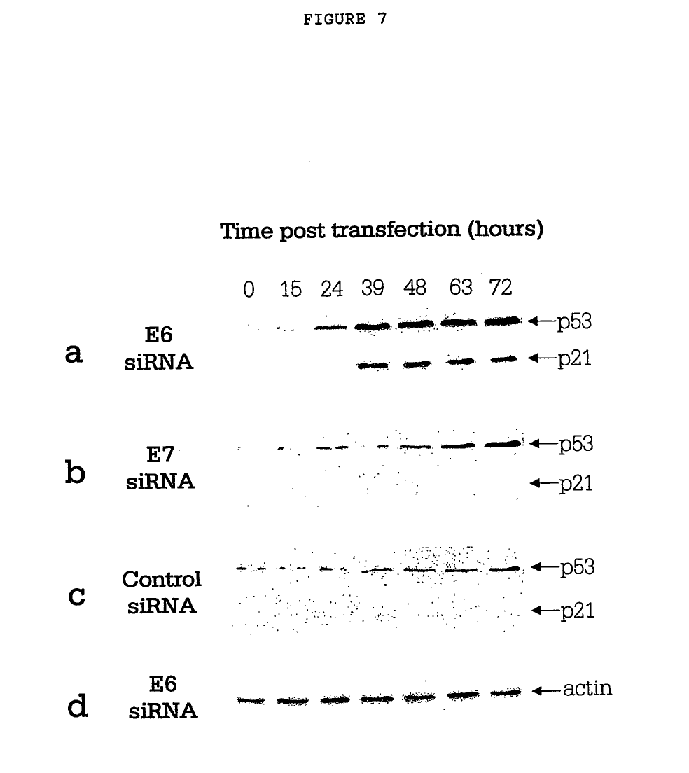

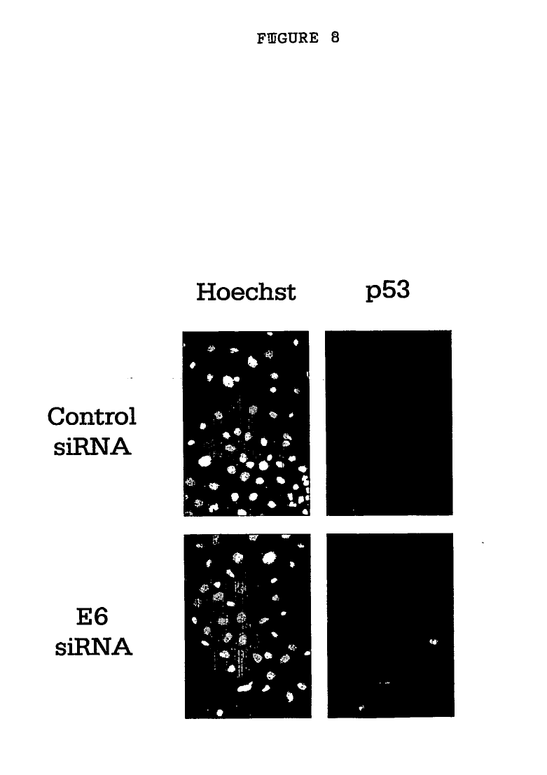

By immunoblotting we observed accumulation of p53 protein after treatment with E6 siRNA (FIG. 7a). The accumulation of p53 was largely nuclear as revealed by indirect immunofluorescence (FIG. 8). Having shown that p53 siRNA levels remain constant in cells treated with E6 siRNA (FIG. 6e) we conclude that the increased p53 protein level (FIG. 7a) represents stabilisation of the p53 protein. However, p53 is a stress response protein and it was also necessary to ascertain that the process of siRNA transfection, by itself, is not sufficient to activate a p53 response. This was investigated by transfecting cells with either control siRNA or E7 siRNA. Although a slight increase in p53 protein levels was observed in both cases, the kinetics were much slower than for cells treated with E6 siRNA and there was no transactivation of p21, a 953 target gene (FIGS. 7b and c). In contrast, stabilisation of p53 in cells treated with E6 siRNA is accompanied by induction of p21 expression (FIG. 7a). Our results thus indicate that p53 becomes stabilised and is activated in E6 siRNA-treated cells. This effect is specific to E6 siRNA and reflects selective E6 gene silencing rather than a generalised stress response.

The p21 protein is a cell cycle inhibitor and induces G1 cell cycle arrest by regulating pRb function (Levine 1997). Although cell growth was reduced in E6 siRNA-treated cells expressing p21, no substantial G1 arrest was observed by FACS analysis (approximately 10% relative to controls). A likely explanation is that p21-mediated effects were compromised due to sustained inactivation of pRb by E7. This implies dominance of E7 protein over E6 siRNA for cell cycle arrest.

E7 Silencing Results in De-Phosphorylation of pRb.

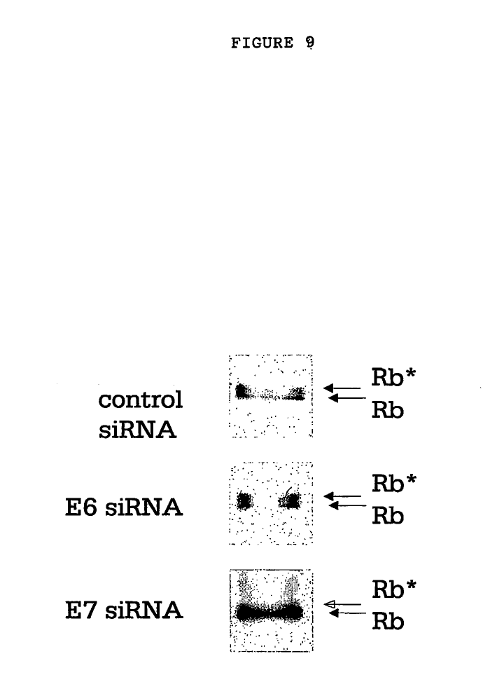

Binding of HPV E7 to pRb and Rb-related cellular proteins results in their hyper-phosphorylation and release of E2F transcription factors. Therefore silencing of HPV E7 may cause reduction or loss of the hyper-phosphorylated form of pRb. This proved to be the case. Treatment of SiHa cells with E7 siRNA resulted in loos of the upper band of pRb which migrates more slowly than the hypo-phosphorylated protein on gel electrophoresis. In contrast, cells treated with either control siRNA or E6 siRNA retained both phosphorylated forms of pRb (as indicated by the doublets of hyper-phosphorylated plus hypo-phosphorylated pRb protein shown in FIG. 9). These observations confirm selective silencing of the HPV E7 gene in cells treated with E7 siRNA.

E7 siRNA Induces Apoptosis of HPY-Positive Cells.

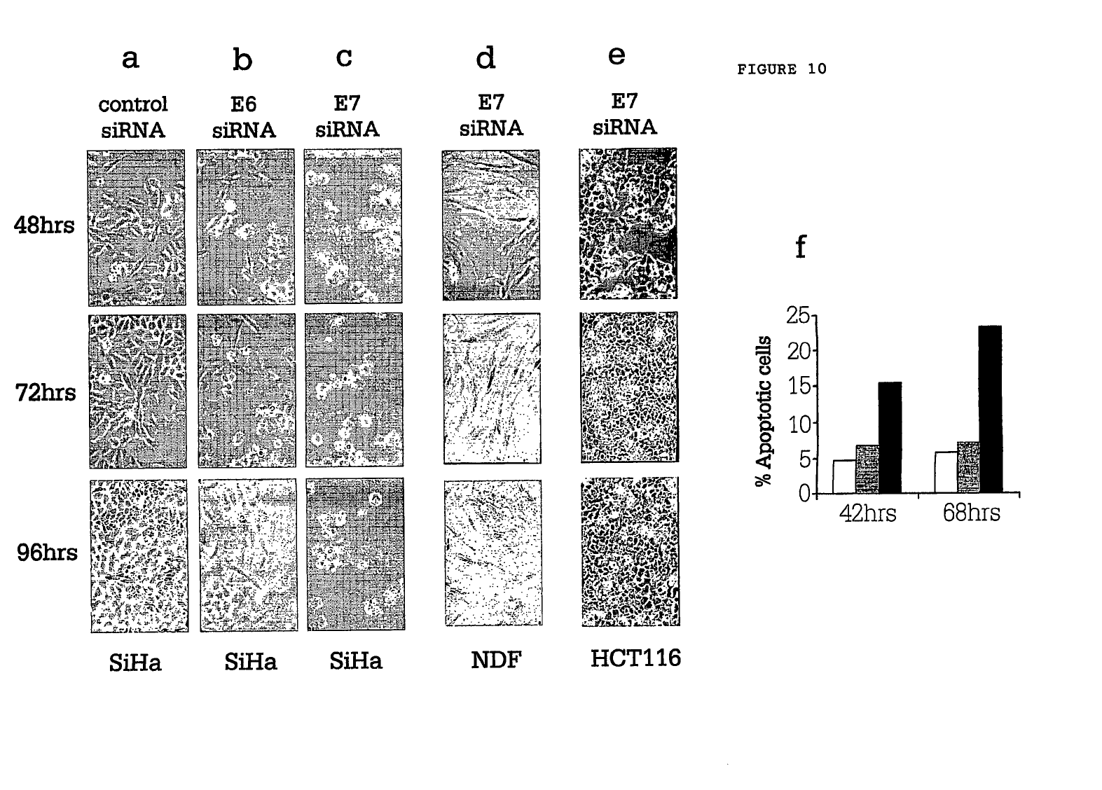

Ideally, a therapeutic agent for use in treatment of human cervical cancer should selectively target the rumour cells for destruction without affecting surrounding normal tissues. In the case of HPV-positive cervical carcinomas this is a realistic objective since the driving force of malignancy is exogenous. Both HPV E6 and E7 are known to influence the cellular apoptotic response (zur Hausen 2000). Having demonstrated the feasibility of selectively silencing these two exogenous viral genes using siRNA (see above) we investigated if viral gene silencing could include selective killing of the HPV-positive cells. Application of E6 siRNA caused cell growth suppression but no significant cell death (FIGS. 10b and f). In contrast E7 siRNA caused the cells to round up and to undergo apoptosis (FIGS. 10c and f).

We considered the possibility that E7 siRNA might induce apoptotic cell death through targeting some hitherto unidentified endogenous gene important for cell viability. However, when E7 siRNA was applied to HPV-negative primary human diploid fibroblasts DF) and human colorectal carcinoma HCTl 16 cells no adverse effects on cell growth or viability were observed (FIGS. 10d and e, and cell growth analyses). Thus we conclude that apoptosis in cells treated with E7 siRNA is initiated by silencing of HPV E7 gene expression and is therefore selective for HPV-positive carcinoma cells.

Others have shown that p53 availability is important for CD95-induced apoptosis in primary human keratinocytes immortalised with E6 and/or E7 and treated with proteasome inhibitor to stabilise p53 (Aguilar-Lemarroy et al 2002). However, meaningful comparison with our present results cannot be drawn since to two experimental systems are fundamentally different. Indeed, a number of conflicting observations on apoptotic effects following blockage of HPV E6 and E7 are difficult to reconcile (zur Hausen 2000). Our present results confirm and greatly extend a previous study using antisense oligonucleotides to target the start codons of HPV-18 E6 and E7: Repeated dosage with E7 antisense caused selective killing of HPV-positive cells, as determined by simple light microscopy, whereas E6 antisense had no apparent effect (Steele 1993). In future studies the application of RNA interference may enable more detailed analysis of E6 and E7 functions at different stages during progression from HPV infection to malignancy.

Discussion

RNA interference, anti-sense RNA and ribozymes all operate at the post-transcriptional level to suppress gene expression. However, the process of RNA interference is several orders of magnitude more efficient than anti-sense or ribozyme strategies (Elbashir et al 2001c). It also consumes high levels of cellular ATP (Nykanen et al 2001). It is therefore possible that RNA interference may cause imbalance within normal cellular biochemical processes and regulatory systems. The present findings indicate that this is not the case. We demonstrate that RNA interference does not block the recovery of endogenous regulatory systems during siRNA-primed silencing of viral genes in human cells. In the case of HPV E6 silencing the p53 protein was stabilised, the p21 cell cycle control gene was expressed and cell growth reduced. E7 silencing, on the other hand, initiated the process of apoptosis (see Results section). Thus we show that cells undergoing RNA interference retain the ability to perform highly complex and biochemically integrated processes involved in differential gene expression and apoptosis. This is an important and novel observation demonstrating that the process of RNA interference does not compromise these critical functions in mammalian cells.

The ability to selectively silence mammalian gene expression using siRNA opens new and exciting routes to the understanding of mammalian cell biology and its pathology. However, it cannot be assumed that all genes will prove equally susceptible to RNA interference. The process is dependent upon mRNA accessibility and, within the target mRNA molecule, upon accessibility of the short internal nucleotide sequence homologous to the siRNA primer. It follows that various factors will influence the vulnerability of a given mRNA to siRNA-mediated degradation, including secondary structures of the mRNA, and proteins which package mRNA for translocation within the cell (Orphanides et al 2002). Other protein-mRNA interactions are also relevant, including proteins which can direct a given mRNA to specific sub-cellular locus (Gu et al 2002), and those mRNAs which can be bound by the proteins they encode, such as p53 (Mosner et al 1995).

We demonstrate that pathogenic viral mRNAs encoded by HPV are vulnerable to RNA interference in mammalian cells. Selective silencing of exogenous viral gene expression by siRNA is particularly relevant to human disease. First, for fundamental research into the pathogenesis of mammalian viruses and for enabling identification of novel therapeutic targets. In addition, siRNA itself may be developed as a novel anti-viral agent to counter viral infection and disease. Being a self-replicative process RNA interference is very efficient. We show that viral gene silencing by a single dose of anti-viral siRNA can be sustained long enough to allow recovery of cellular regulatory systems. In the case of HPV-positive human carcinoma cells this leads to selective killing of the cancer cells.

For therapeutic applications of siRNA, the target mRNA should ideally be recognised via evolutionary conversed nucleotide sequence(s). This minimises potential for loss of homology between the siRNA and the target mRNA due to genetic mutation. Consideration should also be given to the possibility that different cell types may vary in their response to the introduction of short double-stranded siRNA molecules. A particularly apposite example concerns the ability of E6 and E7 proteins to disrupt the expression of interforms and of interferon-inducible genes in efficacy of siRNA-mediated effects observed in the present study.

Our observations indicate that E7 siRNA has major therapeutic potential for the treatment, and possibly prevention of human cervical cancer. We believe that other pathogenic viral agents may similarly be silenced by administration of the relevant siRNAs. The approach of diverse disease where the underlying causes is induced by expression of abnormal gene(s).

Methods

RNA Preparation and mRNA Quantitation

21-nucleotide RNAs (FIG. 5) were synthesised and HPLC purified by MWG (Germany). For annealing of the siRNAs, 20 μM complementary single stranded RNAs were incubated in annealing buffer (20 mM Tris-HCl pH7.5; 10 mM MgCl; and 50 mM NaCl) for 1 min at 90° C. followed by 1 hr at 37° C. For quantitation of mRNA by Northern blotting 0.3 μg of total mRNA, prepared using Oligotex (Qiagen), was run with size markers on a 1% agarose gel at room temperature under standard conditions. HPV16 mRNA was detected using radiolabelled [³²P]-HPV cDNAs as probes and visualised by autoradiography. Total cellular RNA was prepared using the RNeasy kit (Qiagen). For RT-PCR the Reverse-iT one-step kit (Advanced Biotechnologies) was employed. For RT-PCR reactions 0.1 μg total RNA was used. For E6 mRNA amplification, the primers 5′CGGAATTCATGCACCAAAAGAGAACTGCA-3′ and 5′CCCAAGCTTACAGCTGGGTTTCTCTACG-3′ were used in the thermal cycle: 47° C. 30 min; 94° C., 2 min; then 35 cycles of 94° C. 45 sec, 55° C. 45 sec and 72° C. 1 min; followed by 72° C. for 5 min. For E7 mRNA amplification the primers were 5′-CGGAATTCATGCATGGAGATACACCTACAT-3′ and 5′-CGGGAAGCTTATGGTTTCTGAGAACAGATGG-3′, and the thermal cycle as 47° C., 30 min; 94° C., 2 min; then 30 cycles of 94° C. 45 sec, 58° C. 45 sec and 72° C. 2 min; followed by 72° C., 5 min. For p53 mRNA, the primers 5′atggaggagccgcagtcagat3′ and 5′tcagtctgagtcaggcccttc3′ were used, and the thermal cycle as follows: 47° C., 30 min; 94° C. 45 sec, 58° C. 45 sec and 72° C. 2 min; followed by 72° C. for 5 min. For semi-quantitative RT-PCR 100 ng total cellular RNA was diluted 1/20 and 1/400. Northern blots were repeated twice, and semi-quantitative RT-PCRs were repeated two in four times with reproducible results.

Cell Lines and Transfections

CaSKi and SiHa epithelial cell lines are derived from human cervical carcinomas and contain integrated HPV-16 genome, about 600 copies (CaSKi) and 1 to 2 copies (SiHa). CaSKi cells were cultured in RPMI plus 10% foetal calf serum (FCS, Life technologies). SiHa cells were cultured in MEM plus 10% FCS, 1.0 mM sodium pyruvate, and 0.1 mM non-essential amino acids. NDF were cultured in MEM plus 15% FCS, 1.0 mM sodium pyruvate, and 0.2 mM essential amino acids. HCT116 were in DMEM with 10% FCS. All the cell lines were cultured with penicillin 100 units ml⁻¹ and streptomycin 100 μg ml⁻¹ at 37° C. in 5% CO₂ in air. For transfection the cells were trypsinised and subbed into 6 well plates (10 cm²) without antibiotics, 1.5×10⁵ cells per well. After 24 hr the cells were transfected with siRNA formulation into liposomes (Oligofectamine, Life Technologies) according to the manufacturer's instructions. siRNA concentration was 0.58 μg per 1.5×10⁵ cells per will. The final volume of culture medium was 1.5 ml per well. Cells were harvested for analysis at various times thereafter as indicated in the results. Each experiment was carried out four or more times. Transfection efficiencies were established by transfecting cells with liposomes containing FITC-dextran (FD-150; Sigma).

Immunoblotting

Transfected cells were trypsinised, washed in PBS and an aliquot removed for cell counting. The remaining cells were lysed in 50 μl lysis buffer (150 mM NaCl; 0.5% NP40; 50 mM Tris pH 8.0) on ice for 30 min. Samples were diluted 1:1 in 4×strength Laemlli's buffer. Proteins were resolved by 15% SDS-PAGE and electroblotted onto nitrocellulose membrane for antibody detection. Molecular weight markers and purified recombinant human p51 were included as markers as necessary. Monoclonal antibody DO-1 (Oncogene) was used to detect human p53 protein; anti-p21 (SX118) and anti-pRb (G3-245; PharMingen) were used to detect p21 pRb proteins respectively. Actin was detected using polyclonal antibody (Sigma). It was not possible to monitor HPV E6 or E7 protein level since no antibodies are available for their reliable quantitation. Visualisation of bound antibodies was by enhanced chemiluminescence (Roche). Signal quantitation was by scanning signals in the linear range.

Cell Growth, Cell Cycle Analysis and Apoptosis

Cell growth curves were determined by cell counting. For cell cycle analysis the cells were harvested, washed with PBS and fixed in 90% ethanol overnight at −20° C. The fixed cells were pelleted, washed in PBS and resuspended in PBS containing 0.1 μg/ml propidium iodide with 200 U/ml RNase A and analysed by FACS. Apoptotic cells were identified using annesin-V-Fluos (Beobringer) following the manufacturer's protocol.

### REFERENCES

Aguilar-Lemarroy et al (2002) Oncogene 21, 165-175.

Andersson B. S. et al. (1987) Cancer Genet. Cytogenet 24, 335-343

Baker et al (1987) J Virology 61, 962-971.

Bass B. L. (2001) Nature 411, 428-429.

Beer-Romero et al (1997), Oncogene 14, 595-602.

Blume-Jensen P. & Hunter T. (2001) Nature 411, 355-365.

Butz K. et al (1995) Oncogene 10, 927-36.

Butz K. et al (1996) Int. J. Cancer 68, 506-13.

Butz K. (2000) Proc Natl Acad Sci 97, 6693-7.

Caplen N J et al (2001) Proc Natl Acad Sci 98, 9742-9747.

Carthew, R. W. (2001) Curr. Op. Cell. Biol 1, 244-248.

Deininger M. W. N. et al. (2000) Blood 96, 3343-3356.

Deininger M. W. N. et al. (1997) Blood 90, 3691-3698.

Druker B. J. et al. (2001a) N. Engl. J. Med. 344, 1031-1037.

Druker B. J. et al. (2001b) N. Engl. J. Med. 344, 1038-1042.

Elbashir S. M. et al. (2001a) Genes & Dev. 15, 188-200.

Elbashir S. M. et al. (2001b) Nature 411, 494-498.

Elbashir S. M et al (2001c) Nature 411, 428-9

Fire A. et al. (1998) Nature 391, 806-811.

Gong J. G. et al. (1999) Nature 399, 806-809.

Gu et al (2002) J Cell Biol 156, 41-51.

Hamilton A. J.& Baulcombe D. C. (1999) Science 286, 950-952.

Harborth et al (2001) J Cell Sci 114, 4457-4565.

Hengstermann et al. (2001) Proc. Natl. Acad. Sci. USA 98, 1218-1223

Hietanen et al. (2000) Proc Natl Acad. Sci 97, 8501-8506.

Horita M. et al. (2000) J. Exp. Med. 191, 977-984.

Hu et al. (1995) Cancer Gene Ther 2, 19-32.

Hubbard S. R. & Till J. H. (2000) Ann Rev Biochem 69, 373-398.

Ketting et al (2001) Genes Dev 15, 2654-2659.

Kisielow M et al (2002) Biochem J 363, 1-5.

Knight and Bass (2001) Science 293, 2269-2271.

Konopka J. B. et al. (1984) Cell 37, 1035-1042.

Koromilas and Matlashewski (2001) Cytokine Growth Factor Rev, 12, 157-70.

Levine, A. J. (1997) Cell 88, 323-331.

Lipardi et al Cell 107, 297-307.

Liu et al (1999) J Virol 73, 7297-307.

McMurray et al (2001) Int J Exp Pathol 82, 15-33.

McWhirter J. R. & Wang J. Y. (1993) EMBO J. 12, 1533-1546.

McWhirter J. R. et al. (1993) Mol. Cell. Biol. 13, 7587-7595

Minks M. A. et al., (1979) J. Biol. Chem. 254, 10180-10183.

Mosner et al (1995) EMBO J. 14, 4442-4449.

Nykanen et al (2001) Cell 107, 309-321.

Orphanides and Reinberg (2002) Cell 108, 439-451.

Pan et al (1994) Genes Dev 8, 1285-99.

Pan et al (1995) Genes Dev 9, 2157-9.

Patel et al (1999) EMBO J, 18, 5061-5072.

Pim et al (1994) Oncogene 9, 1869-76.

Plasterk R. H. A. & Ketting R. F. (2000) Curr. Op. Genetics & Dev. 10, 562-567.

Scheffner et al (1990) Cell 63, 1129-36.

Scheffner et al (1993) Cell 75, 495-505.

Scheffner et al (1991) Proc. Natl. Acad. Sci. USA 88, 5523-5527.

Sharp P. A. (2001) Genes Dev. 15, 485-490.

Steele et al (1993) Cancer Res 53, 2330-2337

Thomas et al (1999) Oncogene 18, 7690-700.

Veldhoen N & Milner J. (1998) Oncogene 16, 1077-1084.

Venturini et al (1999) Nucleic Acids Res 27, 585-92

Vigneri P. & Wang J. Y. J. (2001) Nature Medicine 7, 228-234.

von Knebel-Doeberitz et al (1992), Int J Cancer 51, 831-4

Wang J. Y. J. (2000) Oncogene 20, 5643-5650.

Zamore P. D. et al., (2000) Cell 101, 25-33.

zur Hausen, H. (2000) J Natl Cancer Inst. 92, 690-8.

1. A method of selective post-transciptional silencing in a mammalian cell of the expression of a viral oncogene comprising introducing into said mammalian cell an siRNA of between 15 and 25 base pairs in length which is homologous to a part of the mRNA sequence of said gene.

2. A method of selective post-transcriptional silencing in a mammalian cell of the expression of the human papilloma virus (HPV) E6 gene comprising introducing into said mammalian cell an siRNA of between 15 and 25 base pairs in length which is homologous to a part of the mRNA sequence of said gene.

3. A method of selective post-transcriptional silencing in a mammalian cell of the expression of the HPV E7 gene comprising introducing into said mammalian cell an siRNA of between 15 and 25 base pairs in length which is homologous to a part of the mRNA sequence of said gene.

4. The method according to claim 3 wherein said siRNA is homologous to an unbroken or contiguous mRNA sequence of said HPV E7 gene.

5. The method according to claim 3 or 4 wherein the HPV E7 gene is an HPV-16 or HPV-18 E7 gene.

6. The method according to claims 2 or 3 wherein HPV is present in the mammalian cell prior to the instruction of said siRNA.

7. The method according to any one of claims 1-4 wherein the siRNA is less than 22 base pairs in length.

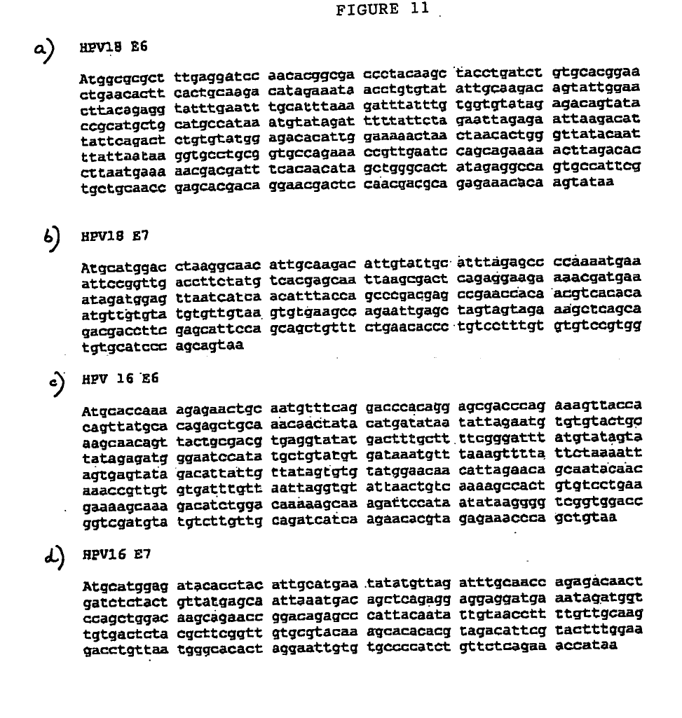

8. An siRNA of between 15 and 25 base pairs in length derived from a nucleic acid molecule selected from the group consisting of:
i) a nucleic acid molecule as represented by any nucleic acid sequence in FIG. 11(a) or
ii) a nucleic acid molecule which hybridizes to any of the nucleic acid sequences in (i) and which has siRNA activity.

9. An siRNA of between 15 and 25 base pairs in length derived from a nucleic acid molecule selected from the group consisting of:
i) a nucleic acid molecule as represented by any nucleic acid sequence in FIG. 11(b) or (d);
ii) a nucleic acid molecule which hybridizes to any of nucleic acid sequences in (i) and which has siRNA activity.

10. The sirRNA according to claim 8 or 9 wherein said RNAi molecule is less than 22 base pairs in length.

11. A vector comprising siRNA according to claim 8 or 9.

12. The vector according to claim 11 wherein said vector is an expression vector adapted for expression of said siRNA.

13. The siRNA of claim 8, 9 or 10, or vector of claim 11 or 12, for use as a medicament.

14. Use of the siRNA of claim 8, 9 or 10, or vector of claim 11 or 12 for the manufacture of a medicament for the treatment of HPV-induced disease.

15. Use according to claim 14 wherein the disease is selected from the group consisting of genital warts; cervical cancer; penile cancer; malignant squamous cell carcinomas; verruca vulgaris.

16. Use of the siRNA of claim 9 for the induction of apoptosis in an HPV-positive cell.

17. Use according to claim 16 wherein the cell is a cervical carcinoma cell.

18. A method of treatment comprising administering to a patient in need of such treatment an effective dose of the siRNA of claim 8, 9 or 10.

19. A pharmaceutical composition comprising the siRNA of claim 8, 9 or 10 and a pharmaceutically acceptable excipient.

20. The pharmaceutical composition of claim 19, comprising an amount of siRNA suitable for treating an HPV-induced disease.

21. The pharmaceutical composition of claim 20, wherein said disease is genital warts, cervical cancer, penile cancer, malignant squamous carcinoma or verruca vulgaris.

22. A pharmaceutical composition comprising the vector of claim 12 and a pharmaceutically acceptable excipient.

23. The pharmaceutical composition of claim 22, comprising an amount of vector suitable for treating an HPV-induced disease.

24. The pharmaceutical composition of claim 23, wherein said disease is genital warts, cervical cancer, penile cancer, malignant squamous carcinoma or verruca vulgaris.

25. A method of inducing apoptosis in an HPV-positive cell comprising exposing said cell to the siRNA of claim 9.

26. The method of claim 25 wherein said cell is a cervical carcinoma cell.

27. A method of treatment comprising administering to a Patient in need of such treatment an effective dose of the siRNA of the vector of claim 12.


SEQUENCE LISTING (25 sequences)

SEQ ID NO: 1 - DNA, 477 bp, Human papillomavirus type 16
atgcaccaaaagagaactgcaatgtttcaggacccacaggagcgacccagaaagttaccacagttatgcacagagctgcaaacaactatacatgatataatattagaatgtgtgtactgcaagcaacagttactgcgacgtgaggtatatgactttgcttttcgggatttatgcatagtatatagagatgggaatccatatgctgtatgtgataaatgtttaaagttttattctaaaattagtgagtatagacattattgttatagtttgtatggaacaacattagaacagcaatacaacaaaccgttgtgtgatttgttaattaggtgtattaactgtcaaaagccactgtgtcctgaagaaaagcaaagacatctggacaaaaagcaaagattccataatataaggggtcggtggaccggtcgatgtatgtcttgttgcagatcatcaagaacacgtagagaaacccagctgtaa

SEQ ID NO: 2 - DNA, 21 bp, Artificial sequence
  siRNA
acugcgacgugagguauautt

SEQ ID NO: 3 - DNA, 21 bp, Artificial sequence
  siRNA
auauaccucacgucgcagutt

SEQ ID NO: 4 - DNA, 21 bp, Artificial sequence
  siRNA
gagguauaugacuuugcuutt

SEQ ID NO: 5 - DNA, 21 bp, Artificial sequence
  siRNA
aagcaaagucauauaccuctt

SEQ ID NO: 6 - DNA, 21 bp, Artificial sequence
  siRNA
augcuguaugugauaaaugtt

SEQ ID NO: 7 - DNA, 21 bp, Artificial sequence
  siRNA
cauuuaucacauacagcautt

SEQ ID NO: 8 - DNA, 21 bp, Artificial sequence
  siRNA
uuuauucuaaaauuagugatt

SEQ ID NO: 9 - DNA, 21 bp, Artificial sequence
  siRNA
ucacuaauuuuagaauaaatt

SEQ ID NO: 10 - DNA, 21 bp, Artificial sequence
  siRNA
cugcgacgugagguauaugtt

SEQ ID NO: 11 - DNA, 21 bp, Artificial sequence
  siRNA
cauauaccucacgucgcagtt

SEQ ID NO: 12 - DNA, 21 bp, Artificial sequence
  siRNA
agaguucaaaagcccuucatt

SEQ ID NO: 13 - DNA, 21 bp, Artificial sequence
  siRNA
ugaagggcuuuugaacucutt

SEQ ID NO: 14 - DNA, 21 bp, Artificial sequence
  siRNA
aggaggaugaaauagauggtt

SEQ ID NO: 15 - DNA, 21 bp, Artificial sequence
  siRNA
ccaucuauuucauccuccutt

SEQ ID NO: 16 - DNA, 477 bp, Human papillomavirus type 18
atggcgcgctttgaggatccaacacggcgaccctacaagctacctgatctgtgcacggaactgaacacttcactgcaagacatagaaataacctgtgtatattgcaagacagtattggaacttacagaggtatttgaatttgcatttaaagatttatttgtggtgtatagagacagtataccgcatgctgcatgccataaatgtatagatttttattctagaattagagaattaagacattattcagactctgtgtatggagacacattggaaaaactaactaacactgggttatacaatttattaataaggtgcctgcggtgccagaaaccgttgaatccagcagaaaaacttagacaccttaatgaaaaacgacgatttcacaacatagctgggcactatagaggccagtgccattcgtgctgcaaccgagcacgacaggaacgactccaacgacgcagagaaacacaagtataa

SEQ ID NO: 17 - DNA, 318 bp, Human papillomavirus type 18
atgcatggacctaaggcaacattgcaagacattgtattgcatttagagccccaaaatgaaattccggttgaccttctatgtcacgagcaattaagcgactcagaggaagaaaacgatgaaatagatggagttaatcatcaacatttaccagcccgacgagccgaaccacaacgtcacacaatgttgtgtatgtgttgtaagtgtgaagccagaattgagctagtagtagaaagctcagcagacgaccttcgagcattccagcagctgtttctgaacaccctgtcctttgtgtgtccgtggtgtgcatcccagcagtaa

SEQ ID NO: 18 - DNA, 477 bp, Human papillomavirus type 16
atgcaccaaaagagaactgcaatgtttcaggacccacaggagcgacccagaaagttaccacagttatgcacagagctgcaaacaactatacatgatataatattagaatgtgtgtactgcaagcaacagttactgcgacgtgaggtatatgactttgcttttcgggatttatgtatagtatatagagatgggaatccatatgctgtatgtgataaatgtttaaagttttattctaaaattagtgagtatagacattattgttatagtgtgtatggaacaacattagaacagcaatacaacaaaccgttgtgtgatttgttaattaggtgtattaactgtcaaaagccactgtgtcctgaagaaaagcaaagacatctggacaaaaagcaaagattccataatataaggggtcggtggaccggtcgatgtatgtcttgttgcagatcatcaagaacacgtagagaaacccagctgtaa

SEQ ID NO: 19 - DNA, 297 bp, Human papillomavirus type 16
atgcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcagaaaccataa

SEQ ID NO: 20 - DNA, 29 bp, Artificial sequence
  Primer
cggaattcatgcaccaaaagagaactgca

SEQ ID NO: 21 - DNA, 28 bp, Artificial sequence
  Primer
cccaagcttacagctgggtttctctacg

SEQ ID NO: 22 - DNA, 21 bp, Artificial sequence
  Primer
atggaggagccgcagtcagat

SEQ ID NO: 23 - DNA, 21 bp, Artificial sequence
  Primer
tcagtctgagtcaggcccttc

SEQ ID NO: 24 - DNA, 30 bp, Artificial sequence
  Primer
cggaattcatgcatggagatacacctacat

SEQ ID NO: 25 - DNA, 31 bp, Artificial sequence
  Primer
cgggaagcttatggtttctgagaacagatgg